(19) United States
(12) Patent Application Publication
(10) Pub. No.: US 2006/0100605 A1
(43) Pub. Date: May 11, 2006

(54) Medical suction tube

(76) Inventors: Mehmet Bicakci, Santa Ana, CA (US); Michael Webo, Mission Viejo, CA (US); Aditya Pandit, Irvine, CA (US)

Correspondence Address: Roger C. Turner, Suite 306, 280 Cagney Ln., Newport Beach, CA, 92663, US

(21) Appl. No.: 10/983,413

(22) Filed: Nov. 6, 2004

(51) Int. Cl. A61M 1/00

(52) U.S. Cl. 604/540; 604/264

(57) ABSTRACT

A medical suction tube tip includes a tubular connector that has a proximal end for engaging the inner diameter of the suction tube, and has a distal end having an inner diameter and an outer diameter. An inner suction tube has a proximal end connected within the inner diameter of the tubular connector, and has a distal end having laterally extending retaining fingers. A coiled wire spring is positioned on the inner suction tube, and is retained between the distal end of the connector and the retaining fingers on the distal end of the inner suction tube. A tubular outer cover encloses the inner suction tube and spring, and has a proximal end connected over the distal end of the tubular connector, and has an enclosed distal end. The cover further includes a plurality of longitudinal flutes extending along the outer surface, and has a plurality of apertures arranged along the flutes. A second embodiment includes the same suction tube, tubular connection, inner suction tube and tubular outer cover. Rather than utilizing a coiled wire spring to obtain the desired weight and flexibility of the suction tube tip, the second embodiment features a plurality of tubular sleeves arranged in alternating stainless steel sleeves and flexible plastic sleeves.

### BACKGROUND OF THE INVENTION

The invention relates to a medical suction tube; particularly to a suction tube having an improved tip.

Suction tubes are used in many surgical and medical procedures to evacuate blood and fluids from a patient. During surgery, blood and other body fluids are commonly removed to permit visual inspection of the surgical site and to recycle the blood or otherwise dispose of the fluids. Suction tubes can be in the form of a hand-held wand or in the form of an extended flexible tube having the proximal end connected to a vacuum or suction source, and having the distal end with openings therein for sucking the fluids into and along the length of the tube. Modern suction tubes are typically produced in sterile packs for one-time use and are then disposable.

Some examples of early prior art devices directed to improved tips for suction devices include U.S. Pat. Nos. 3,963,028; 4,068,664; and 4,767,404.

A current widely-used disposable suction tube is shown in FIG. 1 (PRIOR ART). and has a tip including a tubular adapter 2 having one end for connecting the tip to a flexible suction line 4, and having the other end for connecting an inner suction tube (not shown) extending from the adapter; and having a closely-coiled stainless steel spring 6 enclosing the inner tube and connected to the adapter. The spring has a smooth stainless steel plug 8 attached to the distal end, with the plug having a central bore aligned with the distal end of the inner tube. The inner tube is flexible and has four longitudinal ribs extending to the distal end providing interior channels within the spring to facilitate flow into the distal end of the inner tube.

The foregoing prior art suction tube has several desirable features in that the stainless steel spring provides a desirable weight to the tip and it is easily positioned near the bottom of the cavity being drained. The coils of the spring provide numerous opening for the entry of fluids to flow into and along the interior channels down to the distal end of the inner tube. The spring is also flexible and “springy” for easy maneuvering into passages within the patient and operating field.

The foregoing prior art, spring tip suction tube has an occasional but very serious problem. In certain situations, related to the positioning of the tip within the patient and the flexing of the spring, the patient's internal tissue can become pinched and locked between adjacent coils of the spring. This situation is compounded as the suction tube may also become tangled, and it is very difficult to release the pinched tissue from the tip of the suction tube. When the pinched tip is located in a inaccessible location, it is necessary for the surgeon to cut the suction line to remove the suction force on the tip; and if it can not then be released, the suction tip must be surgically removed, or in extreme situations, be physically pulled out with the patients tissue still retained between the coils; and the area related to the torn tissue must also be treated or repaired.

In view of the foregoing, it is an object of the present invention to provide a suction tube having a smooth tip that can not pinch or damage the tissue of a surgical patient.

It is another object to provide a suction tube having a smooth tip with sufficient weight to orient the tip in a desired downward position near the lowest level of the fluid cavity.

### SUMMARY OF THE INVENTION

The foregoing objects are accomplished by a medical suction tube having an improved suction tube tip comprising: a tubular connector, an inner suction tube, a stainless steel spring over the inner suction tube, all enclosed within a tubular outer cover.

The tubular connector is suitably molded from a medical grade rigid thermoplastic material and has a proximal end having an outer diameter for engaging the inner diameter of the suction tube, and has a distal end having an inner diameter and an outer diameter. The inner suction tube is suitably formed of flexible thermoplastic material, and has a proximal end to be connected within the inner diameter of the tubular connector, and has a distal end having laterally extending retaining fingers.

The spring is positioned on the inner suction tube and the inner suction tube is sealed within the inner diameter of the connector. The spring is retained between the distal end of the connector and the retaining fingers on the distal end of the inner suction tube.

The tubular outer cover encloses the inner suction tube and spring, and has a proximal end with an inner diameter connected over the outer diameter of the distal end of the tubular connector, and has an enclosed distal end. The cover further includes a plurality of longitudinal flutes extending along the outer surface, and has a plurality of apertures arranged in the flutes.

A second embodiment includes the same suction tube, tubular connection, inner suction tube and tubular outer cover. However, rather than utilizing a coiled wire spring to obtain the desired weight and flexibility of the suction tube tip, the second embodiment features a plurality of tubular sleeves. The plurality of tubular sleeves can be a variety of materials, having various lengths and thickness', but are preferably arranged in alternating stainless steel sleeves and flexible plastic sleeves.

The first embodiment and the second embodiment, and the reasonably variations thereof provide an improved suction tube tip having a smooth consistent outer diameter having a weighted distal end.

### BRIEF DESCRIPTION OF THE DRAWINGS

While the novel features of the invention are set forth in the appended claims, the invention will be better understood along with other features thereof from the following detailed description taken in conjunction with the drawings, in which:

FIG. 1 is a side elevational view of a suction tip of the prior art;

FIG. 2 is a side perspective view of the present invention;

FIG. 3 is an exploded side perspective view of a first embodiment of the present invention;

FIG. 4 is a side elevational view of the first embodiment of the present invention;

FIG. 5 is a sectional view taken along 5-5 of FIG. 4;

FIG. 6 is a side perspective view of the interior components of a second embodiment of the present invention;

FIG. 7 is a side elevational view of the of the second embodiment of the present invention; and

FIG. 8 is a sectional view taken along 8-8 of FIG. 7.

### DETAILED DESCRIPTION OF THE INVENTION

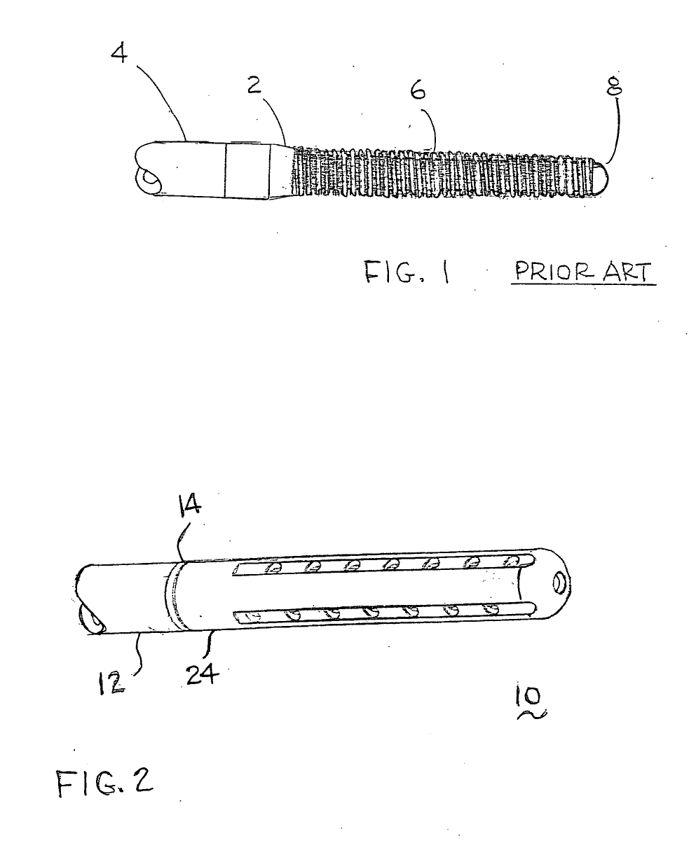

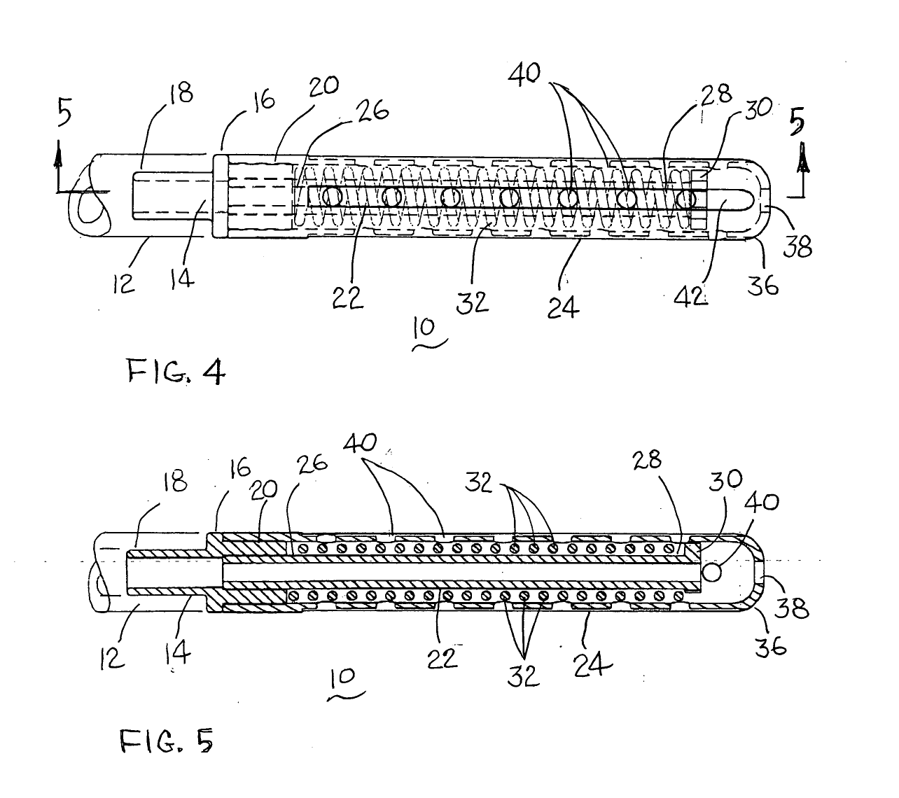

Referring first to FIGS. 2-5, there is illustrated an example of an improved suction tube tip 10 of the present invention. The suction tube tip is typically connected to a suction tube 12 that is about 12 inches in length, formed of a medical grade flexible plastic, suitably poly vinyl chloride (pvc) and having a standard straight tapered connection (clear, molded pvc, not shown) on the proximal end, for further connection to a system including a vacuum or suction source. The suction tube typically has an outer diameter of about 20 French (0.265 inches) and is available in several additional sizes down to about 10 French for pediatric patients.

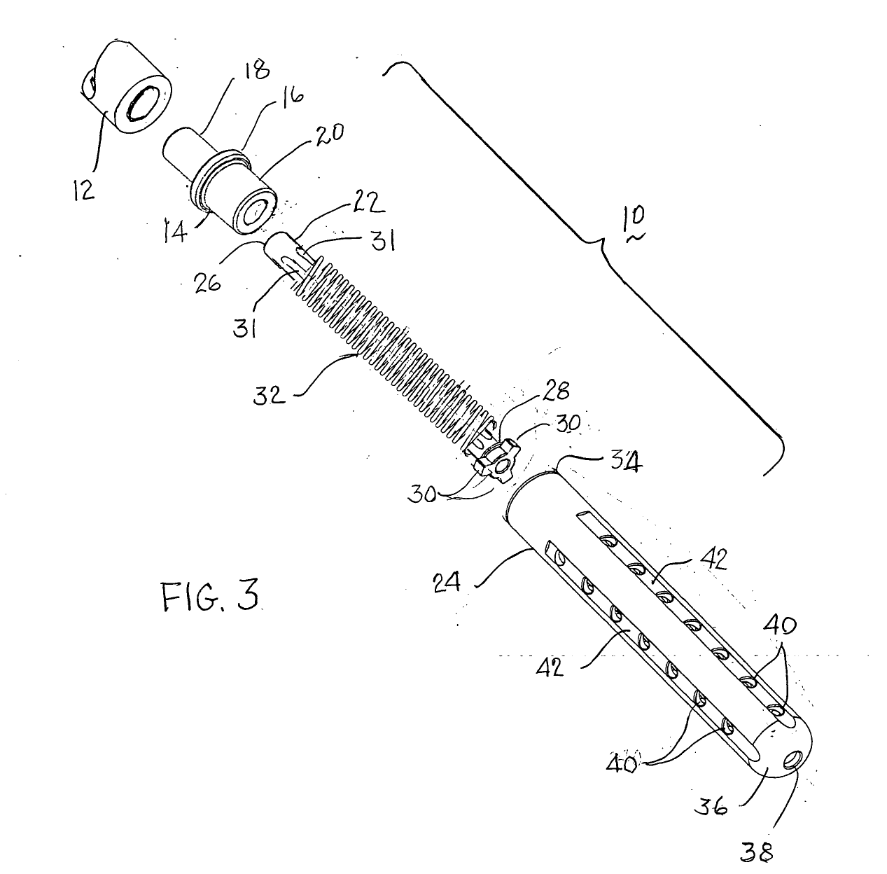

Referring particularly to FIGS. 3-5, a first embodiment of the suction tube tip 10 includes a tubular connector 14 suitably molded of medical grade rigid thermoplastic, about 0.500 inches in length, having a central annular flange 16 about 20 French (0.265 inches) in diameter, corresponding to the outer diameter of the suction tube. The connector has a proximal end 18 having an outer diameter corresponding to the inner diameter of the suction tube (about 0.150 inches) and has a distal end 20 having an inner diameter corresponding to the outer diameter of an inner suction tube 22 (about 0.100 inches), and an outer diameter corresponding to the inner diameter of a tubular outer cover 24 (about 0.200 inches).

The proximal end 18 of the connector 14 is engaged within the flexible suction tube 12 by suitable adhesive, heat and/or sonic welding processes to seal and secure the bonded connection.

The inner suction tube 22 is suitably formed of flexible medical grade thermoplastic (i.e., pvc, polyethylene, etc.) having a length of about 1.500 inches and an outer diameter of about 0.090 inches. The inner suction tube has a proximal end 26 for engaging the inner diameter of the distal end 20 of the tubular connector 14, and has a distal end 28 having a plurality of retaining fingers 30 extending laterally from the distal end. The inner suction tube can further include small longitudinal flutes 31 (see FIG. 3) on the outer surface to facilitate flow along the outer surface to the distal end of the tube.

A closely coiled wire in the form of a spring 32 is utilized to provide the desired weight and flexibility to the suction tube tip 10. A suitable coiled wire or spring is formed from stainless steel wire having a diameter of about 0.001 inches wound into coils having diameters of about 0.100 inches and having a free length of about 1.25 inches. The spring is positioned on the inner suction tube 22, and the proximal end 26 of the inner suction tube is then sealed by suitable adhesive or bonding within the distal end 20 of the tubular connector 14. The spring is securely retained between the proximal end of the connector and the retaining fingers 30 on the distal end of the inner suction tube.

The tubular outer cover 24 is utilized to provide the smooth exterior surface enclosing the inner suction tube 22 and the spring 32. The cover is suitably molded of clear flexible thermoplastic (i.e. pvc), and has an outer diameter of about 20 French (0.265 inches) and a proximal end 34 having an inner diameter of about 0.205 inches connected over the outer diameter at the distal end 20 of the tubular connector 14, and has a rounded enclosed distal end 36. The distal end of the cover has a central aperture 38 (about 0.010 inches in diameter) and the cover includes a plurality of additional apertures 40 (about 0.006 inches in diameter) preferably arranges in about four longitudinal recessed flutes 42 extending along the length of the cover. The recessed flutes allow the suction tip to smoothly be maneuvered within the operating field without snagging by the apertures, and further preclude the apertures from being occluded by contact with the tissue of the patient or surgical debris.

Referring particularly to FIG. 5, a vacuum applied to the suction line 12 creates a suction at the distal end of the inner suction tube 22 within the cover 24, and any fluids in contact with the cover are readily drawn through the apertures 40, around and through the spring 32 to the distal end of the inner suction tube; and are also drawn directly through the aperture 38, and are evacuated at the distal end of the inner suction tube, and transferred along the suction tube 12.

The distal end sub assembly of the spring 32 surrounding the inner suction tube 22 is quite similar in weight, flexibility and function to the spring tip of the prior art. The present invention has the advantageous features of the prior art, and the smooth outer cover 24 eliminates the serious disadvantages of the prior art.

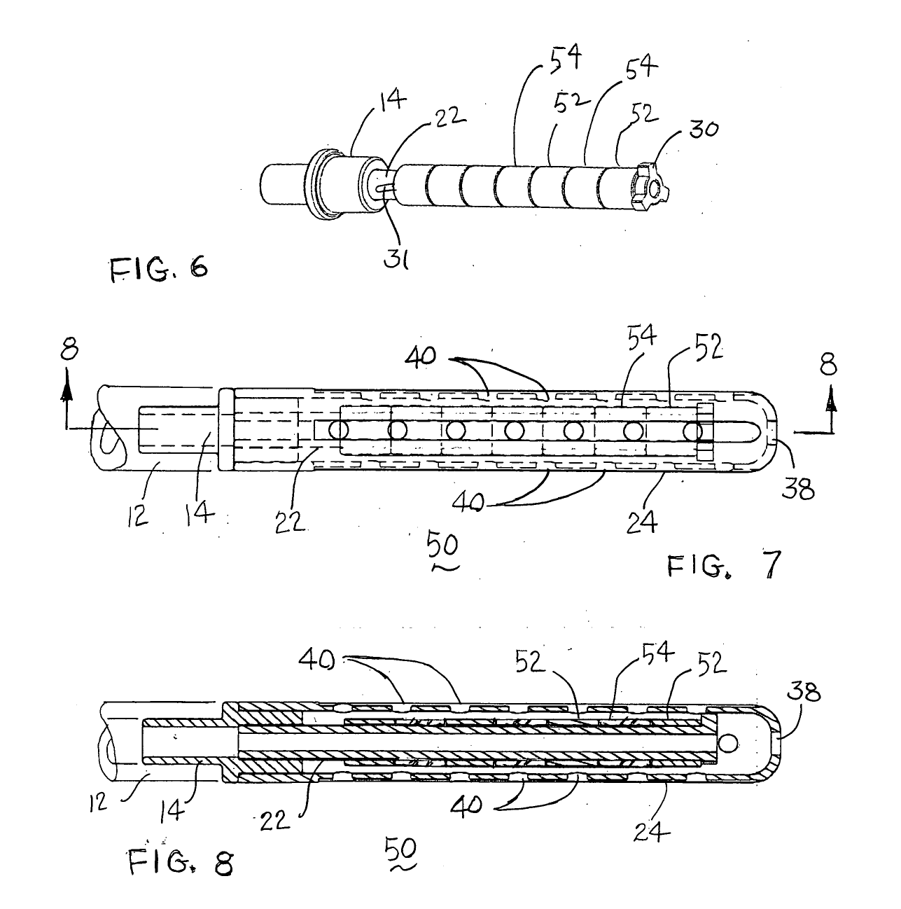

Referring now to FIGS. 6-8, there is illustrated an example of a second embodiment of an improved suction tube tip 50 of the present invention. The second embodiment includes the same suction tube 12, tubular connection 14, inner suction tube 22 and tubular outer cover 24. However, rather than utilizing a coiled wire spring to obtain the desired weight and flexibility of the suction tube tip, the second embodiment features a plurality of tubular sleeves.

As shown particularly in FIG. 6, the tubular sleeves preferably include alternating metallic (suitably stainless steel) sleeves 52 and flexible plastic (suitably pvc) sleeves 54 closely spaced along the inner suction tube 22. Each stainless steel sleeve has a length of about 0.250 inches, a diameter of about 0.100 inches and a wall thickness of about 0.002 inches. The flexible plastic sleeves can be any desired length but are preferable also about 0.250 inches in length, and about 0.100 inches in diameter and have a wall thickness of about 0.002 inches. The alternating stainless steel and plastic sleeves act somewhat like a flexible spring and provide the desired weight so that the suction tube tip can be readily positioned in the downward position near the lowest level of the evacuation cavity of the patient. A variety of materials, sizes and arrangements may be successfully utilized for a desired weight distribution and flexibility of the suction tube tip.

FIG. 7 shows the fully assembled suction tube tip with the outer cover 24 bonded onto the tubular connector 14, and the tip connected to the suction tube 12.

FIG. 8 illustrates the cross section of the suction tube, and more clearly shows how a vacuum applied to the suction line 12 creates a suction at the distal end of the inner suction tube 22 within the cover 24, and any fluids in contact with the cover are readily drawn through the apertures 40, around and through the sleeves 52, 54 to the distal end of the inner suction tube; and are also drawn directly through the aperture 38, and are evacuated at the distal end of the inner suction tube, and transferred along the suction tube 12.

While specific embodiments and examples of the present invention have been illustrated and described herein, it is realized that modifications and changes will occur to those skilled in the art. It is therefore to be understood that the appended claims are intended to cover all such modifications and changes as fall within the spirit and scope of the invention.

1. A medical suction tube having a suction tube tip comprising:
a tubular connector with a proximal end having an outer diameter for engaging the inner diameter of the suction tube, and with a distal end having an inner diameter and an outer diameter;
an inner suction tube with a proximal end connected within the inner diameter of said tubular connector, and with a distal end having a plurality of retaining fingers extending laterally there from;
a coiled wire positioned on said inner suction tube and retained between the distal end of said connector and the retaining fingers; and
a tubular outer cover enclosing the inner suction tube and coiled wire, having a proximal end with an inner diameter connected over the outer diameter of the distal end of said tubular connector, and having an enclosed distal end, and having apertures spaced along the length of said cover.

2. The medical suction tube tip as defined in claim 1, wherein said tubular outer cover further has an aperture in the distal end thereof.

3. The medical suction tube tip as defined in claim 1, wherein said tubular cover further includes a plurality of longitudinal flutes extending along the outer surface thereof and having the apertures arranged along said flutes.

4. The medical suction tube tip as defined in claim 1, wherein said coiled wire is a spring.

5. The medical suction tube tip as defined in claim 1, wherein
said coiled wire is a stainless steel spring, said tubular connector includes a central annular collar having a diameter of about 20 French, said outer cover has an outer diameter of about 20 French, and said tubular cover further includes a plurality of longitudinal flutes extending along the outer surface thereof, and having the apertures arranged along said flutes, so that the suction tube tip provides a smooth consistent outer diameter having a weighted distal end.

6. The medical suction tube tip as defined in claim 1, wherein said inner suction tube further includes longitudinal flutes on the outer surface thereof to facilitate fluid flow along the tube.

7. A medical suction tube having a tip comprising:
a tubular connector with a first end having a diameter for engaging the inner diameter of the suction tube, and with a second end having an inner diameter and an outer diameter;
an inner suction tube with a proximal end connected within the inner diameter of said tubular connector, and with a distal end having a plurality of retaining fingers extending laterally there from;
a plurality of tubular sleeves positioned on said inner suction tube extending from the retaining fingers of the distal end thereof to the second end of said connector; and
a tubular outer cover enclosing the inner suction tube and tubular sleeves, having a proximal end with an inner diameter connected over the outer diameter of the distal end of said tubular connector, and having an enclosed distal end, and having apertures spaced along the length of said cover.

8. The medical suction tube tip as defined in claim 7, wherein said plurality of tubular sleeves includes at least one metallic sleeve.

9. The medical suction tube tip as defined in claim 7, wherein said plurality of tubular sleeves includes at least one plastic sleeve.

10. The medical suction tube tip as defined in claim 7 wherein said plurality of tubular sleeves includes at least ore metallic sleeve and at least one plastic sleeve.

11. The medical suction tube tip as defined in claim 7, wherein said plurality of tubular sleeves are arranged in alternating metallic sleeves and plastic sleeves.

12. The medical suction tube tip as defined in claim 7, wherein said tubular cover further includes a plurality of longitudinal flutes extending along the outer surface thereof and having the apertures arranged along said flutes.

13. The medical suction tube tip as defined in claim 7, wherein said plurality of tubular sleeves are arranged in alternating stainless steel sleeves and pvc plastic sleeves, said tubular connector includes a central annular collar having a diameter of about 20 French, said outer cover has an outer diameter of about 20 French, and said tubular cover further includes a plurality of longitudinal flutes extending along the outer surface thereof and having the apertures arranged along said flutes, so that the suction tube tip provides a smooth consistent outer diameter having a weighted distal end.

14. The medical suction tube tip as defined in claim 7, wherein said inner suction tube further includes longitudinal flutes on the outer surface thereof to facilitate fluid flow along the tube.
